SEQUENCE LISTING (10 sequences)

SEQ ID NO: 1 - DNA, 19 bp, Staphylococcus aureus
ggcctgcacaaggacgtct

SEQ ID NO: 2 - DNA, 23 bp, Staphylococcus aureus
actgcggaggctaactatgtcaa

SEQ ID NO: 3 - DNA, 28 bp, Staphylococcus aureus
acaaycyrwtttttagttttatttatga

SEQ ID NO: 4 - DNA, 27 bp, Staphylococcus aureus
attaacacaacccgcatcatttgatgt

SEQ ID NO: 5 - DNA, 24 bp, Staphylococcus aureus
ggcccatacaccaagatagacatc

SEQ ID NO: 6 - DNA, 25 bp, Staphylococcus aureus
gggccaatccttcggaagatagcat

SEQ ID NO: 7 - DNA, 22 bp, Staphylococcus aureus
gttccagacgaaaaagcaccag

SEQ ID NO: 8 - DNA, 26 bp, Staphylococcus aureus
ggctcaggtactgctatccaccctca

SEQ ID NO: 9 - DNA, 22 bp, Staphylococcus aureus
gtaacgttgtaaccaccccaag

SEQ ID NO: 10 - DNA, 24 bp, Staphylococcus aureus
gaacctctgctcaacaagttccag

(19) United States
(12) Patent Application Publication
(10) Pub. No.: US 2015/0159199 A1
(43) Pub. Date: Jun. 11, 2015

(54) Methods and Systems for the Detection of Methicillin Resistant Staphylococcus Aureus

(71) Applicant: Eng Lee Tan, Singapore (SG)

(72) Inventor: Eng Lee Tan, Singapore (SG)

(73) Assignee: INNOMART PTE LTD, Singapore (SG)

(21) Appl. No.: 14/385,566

(22) PCT Filed: Mar. 22, 2012
(86) PCT No.: PCT/SG2012/000096

§ 371 Date: Feb. 11, 2015

Related U.S. Application Data
(60) Provisional application No. 61/611,587, filed on Mar. 16, 2012.

(51) Int. Cl. C12Q 1/68

(52) U.S. Cl. CPC C12Q 1/689; C12Q 2600/158

(57) ABSTRACT

Described according to an embodiment of the invention is a method and a system thereof, for the detection of at least one of Staphylococcus aureus (SA), the antibiotic resistant forms thereof, and the antibiotic resistant forms of other bacteria. The method comprises obtaining a sample, the extraction of nucleic acids from the sample and bringing the extracted nucleic acids into contact with a first primer pair complementary to a targeted segment of the attBscc gene, a second primer pair complementary to a targeted segment of the mecA gene, and a third primer pair complementary to a targeted segment of the orfX gene.

### TECHNICAL FIELD

Aspects of the present disclosure relate to techniques for the detection of Staphylococcus aureus (SA), methicillin resistant Staphylococcus aureus (MRSA) and methicillin resistant bacteria that are non SA and uses thereof for diagnostic purposes.

### BACKGROUND

Methicillin resistant Staphylococcus aureus (MRSA) infection is a serious problem faced by hospitals all over the world. Outbreaks can result easily from an infected individual patient, and cannot be contained easily by antibiotics. Immuno-suppressed individuals as well as young children, and elderly persons are the most susceptible to the risks of such an infection, which can result is death within hours of first exposure to the bacteria. Thus, early detection of an infected individual is crucial for both effective patient management and containment of an outbreak.

In U.S. Pat. No. 7,838,221, there is disclosed SCCmec right extremity junction (MREJ) sequences for the detection and/or identification of MRSA, and Staphylococcal chromosomal cassette (SCCmec), methicillin/oxacillin resistance (mecA) and open reading frame of unknown function (OrfX) are targeted regions for the detection of MRSA and SA. In US 2008/0038737 A1, methods and apparatus for carrying out multiple amplification reactions is a single reaction chamber are disclosed, and in which the mecA, OrfX and Staphylococcal protein A (spa) regions are targeted. The commercial assay kit namely Xpert™ MRSA/SA SSTI Assay (Cepheid) target the spa, mecA and SCCmec sites and can only identify MRSA and SA, albeit simultaneously. Similar current methods also cannot be applied for direct detection of either MRSA, SA or even other strains of methicillin-resistant bacteria from nonsterile specimens without the previous isolation, capture or enrichment of MRSA or SA. There is hence an unmet need for a fast and effective test kit which can differentiate MRSA from both non-MRSA as well as other bacteria that are MR.

### SUMMARY

In accordance with a first aspect of the disclosure, there is disclosed a process for the detection of at least one of Staphylococcus aureus (SA), the antibiotic resistant forms thereof, and the antibiotic resistant forms of other bacteria comprising obtaining a sample, the extraction of nucleic acids from the sample and bringing the extracted nucleic acids into contact with a first primer pair complementary to a targeted segment of the attBscc gene, a second primer pair complementary to a targeted segment of the mecA gene, and a third primer pair complementary to a targeted segment of the OrfX gene. There is further an amplification of the targeted segment in each of the attBscc, OrfX and mecA genes to an extent that would be sufficient for detection of a presence of the targeted segment if at least one of the three genes are present in the extracted nucleic acids from said sample, followed by the detection of the presence of the targeted segments of nucleic acids to determine the presence of as least one of Staphylococcus aureus (SA), the antibiotic resistant forms thereof, and the antibiotic resistant forms of other bacteria in the sample.

In accordance with a second aspect of the disclosure, there is disclosed one or more portions of a test kit for the detection of the presence of at least one of SA, the antibiotic resistant forms thereof, and the antibiotic resistant forms of other bacteria in nucleic acids extracted from a sample. The kit includes means for exposing the extracted nucleic acids with a first primer pair complementary to a targeted segment of the attBscc gene, a second primer pair complementary to a targeted segment of the mecA gene, and a third primer pair complementary to a targeted segment of the orfX gene.

### DESCRIPTION OF DRAWINGS

FIG. 1 is a diagram showing fluorescent emission levels corresponding to amplified products at 520 nm.

FIG. 2 is a diagram showing fluorescent emission levels corresponding to amplified products at 556 nm.

FIG. 3 is a diagram showing fluorescent emission levels corresponding to amplified products at 615 nm.

FIG. 4 is a diagram showing as embodiment of representative real-time PCR running conditions.

### DETAILED DESCRIPTION

Reference will now be made in detail to particular aspects of a representative set of embodiments of the present disclosure, examples of which are illustrated in the accompanying drawings. While the disclosure will be described in conjunction with a number of embodiments, it will be understood that such embodiment(s) are not intended to limit the scope of the invention to the particular embodiments. On the contrary, the disclosure is intended to cover alternatives, modifications and equivalents, which may be included within the spirit and scope of the invention as defined by the appended claims. Furthermore, in the following detailed description of embodiments in accordance with the present disclosure, numerous specific details ate set forth in order to provide a thorough understanding of the present invention. However, it will be recognized by one of ordinary skill in the art that the present invention may be practiced without these specific details. In other instances, well-known methods, procedures and components have not been described in detail as not to unnecessarily obscure aspects of embodiments of the present invention.

Staphylococcus aureus (SA) is a clinically significant pathogen that causes a wide spectrum of clinical manifestations, such as pneumonia, wound infections, septicemia and endocarditis. Beta-lactam antimicrobial agents are often used as the preferred drags for serious SA infections. However, since the launch of methicillin in 1961, methicillin-resistant Staphylococcus aureus (MRSA) strains have evolved into notorious community pathogens known to cause serious hospital infections worldwide (Jevons, 1961). SA acquires methicillin resistance by insertion into the chromosome of a mobile generic element, Staphylococcal cassette chromosomes (SCCs). SCCs are relatively large fragments of DNA that always insert into the orfX gene on the SA chromosome. SCC can encode antibiotic resistance and/or virulence determinants, including the methicillin resistance gene (mecA). SCCs can be classified into Staphylococcal cassette chromosome mec (SCCmec) or non-SCCmec groups. The mecA gene encodes as additional low-affinity penicillin-binding protein, PBP2a, which is not inhibited by existing Beta-lactam antibiotics, i.e. resistance to B-lactam antibiotics is maintained by production of PBP2a, which fails to bind methicillin and other B-lactam antibiotics (Hiramatsu et al., 2001). Further, integration and excision of SCCmec by recombinases occur within a specific attachment sits (attBscc) on the SA chromosome at the 3′ end of the orfX (Hiramatsu et al., 2001).

The present invention has been developed to provide a rapid and effective diagnosis of MRSA to facilitate or effectuate improved patient management. In addition to the more recent aforementioned assays for detection of MRSA, numerous molecular approaches which reduce the time for diagnosis of MRSA have been described in the following literature: Baron, 1995; Francois et al., 2003; Grisold et al., 2002; Jonas et al., 2002; Kearns et al., 1999; Reischl et al., 2000; Shresthat et al., 2002; Tan et al., 2001.

Disclosed herein are novel DNA sequences relating to primers and probes which are specific to particular target regions within the attBscc, orfX and mecA genes of SA. The attBscc site is found in OrfX, and is a highly conserved region across SA strains (Hiramatsu, 2001). attBscc contains a 15-bp sequence that, when SCCmec is integrated in the chromosome, is present at the chromosome-SCCmec junctions. Thus, attBscc is a site that is potentially highly specific to MRSA but does not appear to be exploited in current assays for the detection of MRSA. Other aspects of the present disclosure relate to further novel primers and probes derived from the novel DNA sequences. Also disclosed herein are processes for detecting the presence or absence of an MRSA strain in a sample comprising a mixed culture, such as a non-sterile specimen without a previous isolation, capture, or enrichment of one or more bacteria strains within.

### Design and Synthesis of Primers and Probes

The DNA sequences in the attBscc, mecA and OrfX genes of the SA genome were evaluated for suitable oligonucleotides for hybridization or PCR amplification by computer software analysis. Potential candidate oligonucleotides for the primers and probes are evaluated and designed for specificity by BLAST analysis on the NCBI website, and for optimal sequence length using Lasergene 7 software so as to prevent unwanted features such as secondary structures. A set of primers and probes for each of the 3 target regions attBscc, mecA and OrfX genes were designed to detect all 5 reported sub-types of MRSA namely NCTC 10442, N315, 85/3907, CA05 and WIS, as shown in Table 1. “Y”, “R” and “W” present in the primers and probes in Table 1 each represents the degenerate bases which can be substituted with C or T; A or G; and A or T respectively. In some embodiments, the oligonucleotide sequences used for the primers and probes are as shown in Table 1, and listed as SEQ IDs NO. 1-10 in the Sequence Listing included herewith. In souse other embodiments, an oligonucleotide sequence comprising a fragment of at least 19 nucleotides of each of the corresponding primers and probes are used in the detection of the target regions attBscc, mecA and orfX.

Each set comprising a forward primer, a reverse primer and a probe which are complementary to each of the mecA and orfX sites are designed. In the case of the attBscc site, a set comprising a forward primer, two reverse primers and a probe which are complementary to the attBscc site are designed. The approximate amplicon size for each of the attBscc, mecA and orfX target regions corresponding to each of the five reported MRSA sub-types are as shown in Table 2. Three different fluorescent dyes are selected and each are used for the labelling of the probe targeting a specific region. Each probe is labeled with a different fluorescent dye at the 5′ end. As each fluorescent dye emits fluorescence at a particular wavelength (channel), the simultaneous detection and differentiation of MRSA from other MR-microorganisms or non-methicillin resistant SA in a mixed culture sample is made possible.

TABLE 1

| Primer/ | |
|---|---|
| Probe | Nucleotide Sequence (5′-3′) |
| AttBscc | GGCCTGCACAAGGACGTCT |
| FP | |
| AttBscc | ACTGCGGAGGCTAACTATGTCAA |
| RP1 | |
| AttBscc | ACAAYCYRWTTTTTAGTTTTATTTATGA |
| RP2 | |
| AttBscc | [FAM] ATTAACACAACCCGCATCATTTGATGT [BHQ1] |
| Probe | |
| mecA FP | GGCTCAGGTACTGCTATCCACCCTCA |
| mecA RP | GTAACGTTGTAACCACCCCAAG |
| mecA | [HEX] GAACCTCTGCTCAACAAGTTCCAG [BHQ1] |
| Probe | |
| OrfX FP | GGCCCATACACCAAGATAGACATC |
| OrfX RP | GGGCCAATCCTTCGGAAGATAGCAT |
| OrfX | [Texas Red] GTTCCAGACGAAAAAGCACCAG [BHQ2] |
| Probe | |

Y-C/T, R-A/G, W-A/T

TABLE 2

| Target site | Type(s) | Strain(s) | Amplicon size |
|---|---|---|---|
| attBscc | I | NCTC 10442 | 185 |
| | II | N315 | 287 |
| | II | 85/3907 | 165 |
| | IV | CA05 | 287 |
| | V | WIS | 165 |
| mecA | I to V | All of the above five strains | 299 |
| orfX | I to V | All of the above five strains | 187 |

### Samples

Samples may comprise but are not limited to: any clinical sample, any environmental sample, any microbial culture, any microbial colony, any tissue and any cell line.

### Preparation of Sample

In some embodiments, DNA is extracted from the sample supplied using the QIAamp® Dneasy Blood and Tissue mini kit, as per the manufacturer's instructions. Other non-limiting methods of DNA extraction may also be used, as described in “PCR Protocols, A Guide To Methods and Applications” (ed. Innis, Academic Press, N.Y. 1990) or any other DNA extraction method generally practised by an ordinary person skilled in the art.

### DNA Amplification

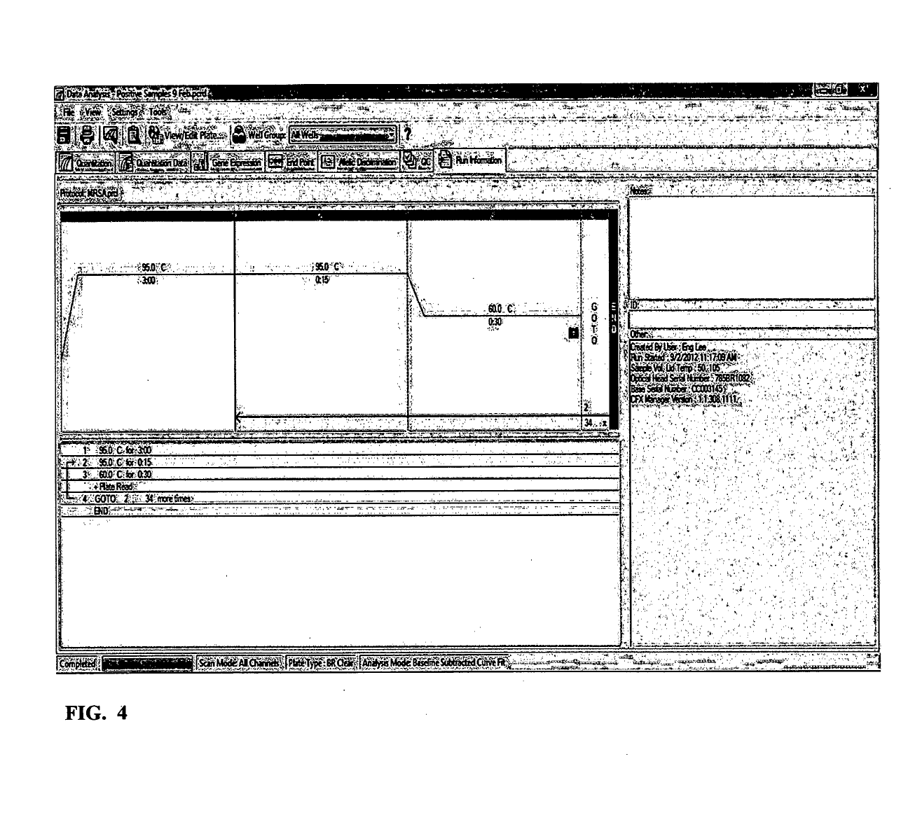

In some embodiments, real-time PCR analysis is used to amplify the nucleic acids is the sample. Multiplex real-time PCR analysis can be carried out according to the manufacturer's instructions using the CFX96 Real-time PCR Detection system. In as embodiment, the specific PCR thermocycling conditions comprise 95° C. for 3 mins, followed by 35 cycles of 95° C. for 15 seconds and 60° C. for 30 seconds on the CFX96 Real-Time PCR Detection System, of which a graphical representation of the above-mentioned protocol steps is shown in FIG. 4. The components of the real-time PCR set-up are as shows in Table 3:

TABLE 3

| | Volume |
|---|---|
| Reagent | (per reaction tube, μl) |
| Reaction mix | 25 |
| attBscc FP (10 uM) | 0.75 |
| attBscc RP1 (10 uM) | 0.75 |
| attBscc RP2 (10 uM) | 0.75 |
| attBscc probe (10 uM) | 0.75 |
| orfx FP (10 uM) | 0.5 |
| orfx RP (10 uM) | 0.5 |
| orfx probe (10 uM) | 0.5 |
| mecA FP (10 uM) | 1 |
| mecA RP (10 uM) | 1 |
| mecA probe (10 uM) | 1 |
| IC (internal control) FP | 0.5 |
| IC (internal control) RP | 0.5 |
| IC (internal control) Probe | 0.5 |
| IC (internal control) DNA | 1 |
| Nuclease~free water | 13 |
| DNA Template | 2 |
| Total | 50 |

### Detection of Nucleic Acids

The detection of nucleic acids can be achieved via utilising a probe technology such as the TaqMan® probe technology where the probe of the target region is labeled with a reporter dye and a quencher. In some embodiments, the attBscc probe is labeled with 6-FAM (520 nm) at the 5′ end and Black Hole Quencher (BHQ) 1 at the 3′ end; the mecA probe is labeled with HEX (556 nm) at the 5′ end and BHQ 1 at the 3′ end; and the OrfX probe is labeled with Texas Red (615 nm) at the 5′ end and BHQ 2 at the 3′ end, as shown in Table 1. In other embodiments, the attBscc probe, mecA probe and orfX probe may each be labeled with a different fluorescent dye, i.e. 3 different fluorescent dyes, in which each of the fluorescent dye is compatible with the corresponding probe. Upon exonuclease digestion during the PCR reaction cycles, the fluorescent dye is released from the quenched system and the fluorescence emitted is detected by the fluorimeter. In the embodiments where the attBscc, mecA and orfX probes are labeled with the 6-FAM, HEX and Texas Red fluorescent dyes respectively, the detection of the 6-FAM, HEX and Texas Red fluorescences is at the 520 nm, 556 and 615 wavelengths respectively. In other embodiments, depending on the other types of fluorescent dyes which have been used, the detection of fluorescence emission is to be conducted in the corresponding wavelength channel indicated for the fluorescent dye, as per the specific manufacturer's instructions.

The type of combination of emission(s) detected at the different wavelengths of the fluorescent dye tagged to the specific probe will determine the type(s) of bacteria strain(s) present in the sample tested, as shown in Tables 4A and 4B. The detection of fluorescence emission in all the 3 different wavelength channels will indicate the presence of MRSA in a mixed culture sample, in the absence of MRSA, the detection of an emission signal only in the 615 nm wavelength channel will indicate the presence of non-methicillin resistant SA, while the detection of an emission signal only in the 556 nm wavelength channel will indicate the presence of other methicillin resistant bacteria strains that are non-SA. Table 5 as shown below, is a table of results from an embodiment of the PCR step of the method, and proves the effectiveness in the detection of MRSA in a known pure MRSA sample (represented by A02) and known mixed culture samples of MRSA with SA, and MRSA with S. epidermidis (represented by A03 and A04 respectively). Table 5 also shows the effectiveness in the detection of a known sample of pure SA that is non-methicillin resistant (represented by A01) and the detection of a known sample of a bacteria strain that is methicillin resistant such as S. epidermidis (represented by A06). In the case of a bacteria strain such as a known sample of E. coli (represented by A05) which is neither methicillin resistant nor a SA strain, there is no signal in any of the wavelength channels.

TABLE 4A

| | | | Other methicillin |
|---|---|---|---|
| Probe (flourescence | non-methicillin | | resistant bacteria |
| emission wavelength) | resistant SA | MRSA | strains (non-SA) |
| attBscc probe (520 nM) | | + | |
| mecA probe (556 nM) | | + | + |
| orfx probe (615 nM) | + | + | |

Where “+” indicates emission will be detected for that bacteria strain

TABLE 4B

| | Wavelength of detected |
|---|---|
| Bacteria strain type | flourescence |
| Non-methicillin resistant SA | 615 nm only |
| MRSA | 520 nm, 556 nm and 615 nm |
| Other methicillin resistant | 556 nm only |
| bacteria strain (non SA) | |

TABLE 5

| | | | | | | | |
|---|---|---|---|---|---|---|---|
| | | | | | Thresh- | | |
| | | | | | old | | C(t) |
| | | Con- | | | Cycle | C(t) | Std. |
| Well | Fluor | tent | Target | Sample | (C(t)) | Mean | Dev |

| | | | | | | |
|---|---|---|---|---|---|---|
| A01 | FAM | Unkn | SA | N/A | 0.00 | 0.000 |
| A02 | FAM | Unkn | MRSA | 23.92 | 23.92 | 0.000 |
| A03 | FAM | Unkn | MRSA + SA | 24.96 | 24.96 | 0.000 |
| A04 | FAM | Unkn | MRSA + SE | 24.99 | 24.99 | 0.000 |
| A05 | FAM | Unkn | EC | N/A | 0.00 | 0.000 |
| A06 | FAM | Unkn | SE | N/A | 0.00 | 0.000 |
| B12 | FAM | NTC | | N/A | 0.00 | 0.000 |
| A01 | HEX | Unkn | SA | N/A | 0.00 | 0.000 |
| A02 | HEX | Unkn | MRSA | 23.10 | 23.10 | 0.000 |
| A03 | HEX | Unkn | MRSA + SA | 24.17 | 24.17 | 0.000 |
| A04 | HEX | Unkn | MRSA + SE | 24.20 | 24.20 | 0.000 |
| A05 | HEX | Unkn | EC | N/A | 0.00 | 0.000 |
| A06 | HEX | Unkn | SE | 34.84 | 34.84 | 0.000 |
| B12 | HEX | NTC | | N/A | 0.00 | 0.000 |
| A01 | Texas Red | Unkn | SA | 31.44 | 31.44 | 0.000 |
| A02 | Texas Red | Unkn | MRSA | 21.25 | 21.25 | 0.000 |
| A03 | Texas Red | Unkn | MRSA + SA | 22.25 | 22.25 | 0.000 |
| A04 | Texas Red | Unkn | MRSA + SE | 22.30 | 22.30 | 0.000 |
| A05 | Texas Red | Unkn | EC | N/A | 0.00 | 0.000 |
| A06 | Texas Red | Unkn | SE | N/A | 0.00 | 0.000 |
| B12 | Texas Red | NTC | | N/A | 0.00 | 0.000 |

Detection of targeted regions may include other methods such as, but are not limited to, molecular beacons, amplifluors, chemiluminescence, mass spectrometry, etc.

### Representative Example 1

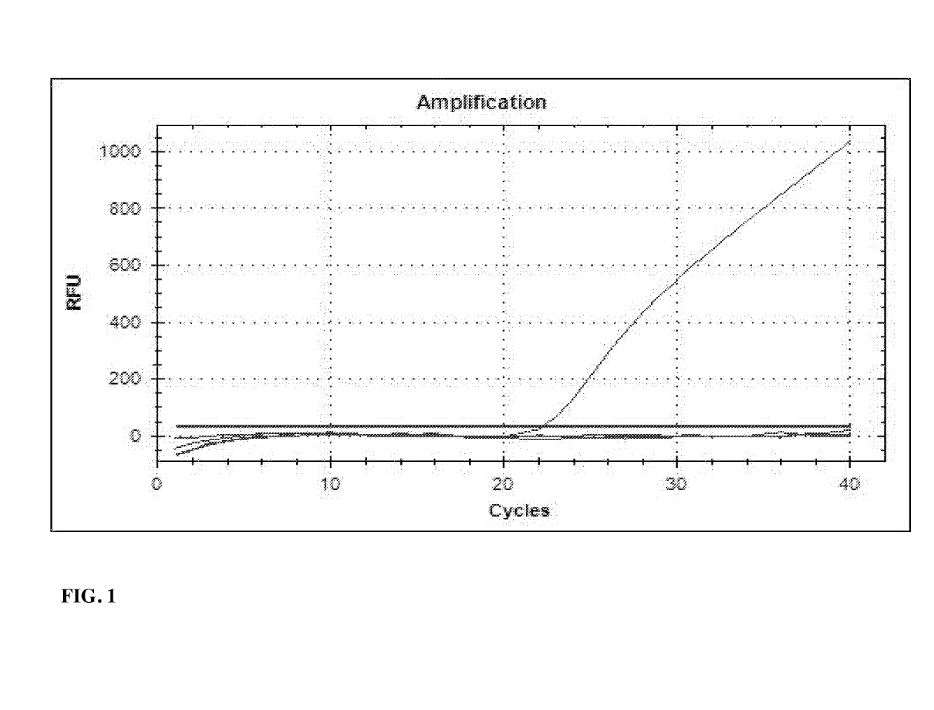

As shown in FIG. 1, the primers and probe targeting the attBscc site in the multiplex assay are specific for detecting MRSA in the fluorescent channel 520 nm, in the presence of non-methicillin resistant SA, E. coli and S. epidermidis as controls.

### Representative Example 2

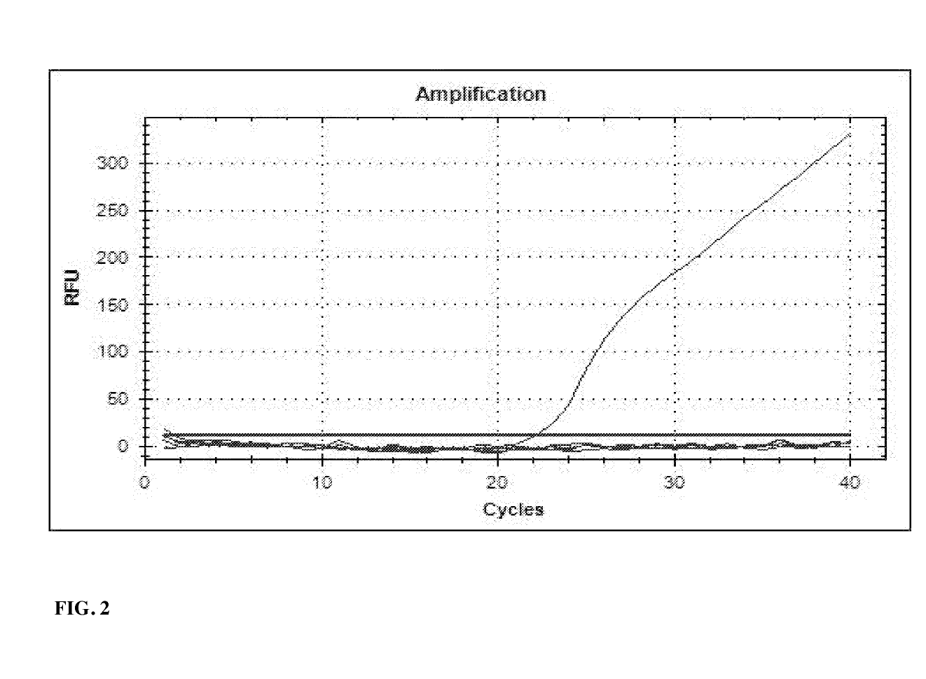

As shown in FIG. 2, the primers and probe targeting the mecA site in the multiplex assay are specific for detecting MRSA in the fluorescent channel 556 nm, in the presence of non-methicillin resistant SA, E. coli and S. epidermidis as controls.

### Representative Example 3

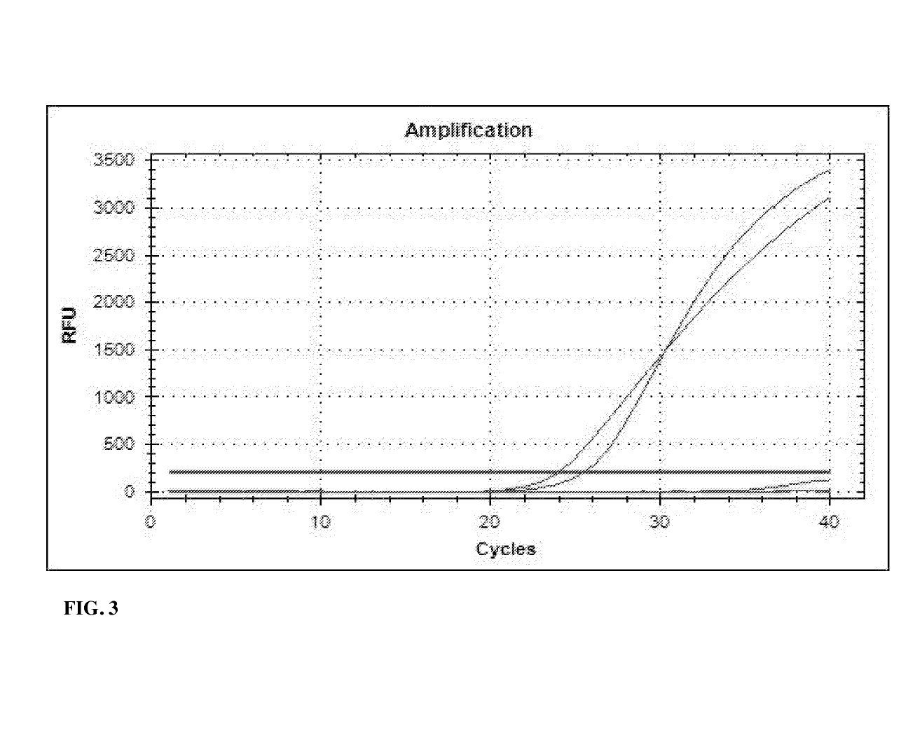

As shown is FIG. 3, the primers and probe targeting the orfX site in the multiplex assay are specific for detecting SA in the fluorescent channel 615 nm, in the presence of E. coli and S. epidermidis as controls.

### REFERENCES

(1) US Food and Ding Administration. 510(k) substantial equivalence determination decision summary assay and instrument combination template. Retrieved from: http://www.accessdata.fda.gov/cdrh_docs/reviews/K080837.pdf

(2) Baron, E. J. 1995. Genetic aspects of methicillin resistance in Staphylococcus aureus and methods used for its detection in clinical laboratories in the United States. J. Chemother. 7 (Suppl. 3):87-92.

(3) François, P., D. Pittet, M. Bento, B. Pepey, P. Vaudaux, D. Lew, and J. Schrenzel. 2003. Rapid detection of methicillin-resistant Staphylococcus aureus directly from sterile or nonsterile clinical samples by a new molecular assay. J. Clin. Microbiol. 41:254-260.

(4) Grisold, A. J., E. Leitner, G. Muhlbauer, E. Marth, and H. H. Kessler. 2002. Detection of methicillin-resistant Staphylococcus aureus and simultaneous confirmation by automated nucleic acid extraction and real-time PCR, J. Clin. Microbiol. 40:2392-2397.

(5) Hiramatsu, K., L. Cui, M. Kuroda, and T. Ito. 2001. The emergence and evolution of methicillin-resistant Staphylococcus aureus. Trends Microbiol. 9:486-493.

(6) Innis, M. A., D. H. Gelfand, J. J. Sninsky, and T4. White. 1990. PCR protocols: A guide to method and applications. Academic Press, New York.

(7) Jevons MP. ‘Celbenin’-resistant staphylococci. BMJ. 1961;1:124-125.

(8) Jonas, D., M. Speck, F. D. Daschner, and H. Grundmann. 2002. Rapid PCR-based identification of methicillin-resistant Staphylococcus aureus from screening swabs. J. Clin. Microbiol. 40:1821-1823

(9) Kearns, A. M., P. R. Seiders, J. Wheeler, R. Freeman, and M. Steward. 1999. Rapid detection of methicillin-resistant staphylococci by multiplex PCR. J. Hosp. Infect. 43:33-37.

(10) Reischl, U., H. J. Linde, M. Metz, B. Leppmeier, and N. Lehn. 2000. Rapid identification of methicillin-resistant Staphylococcus aureus and simultaneous species confirmation using real-time fluorescence PCR. J. Clin. Microbiol. 38:2429-2433.

(11) Shrestha, N. K., M. J. Tuohy, G. S. Hall, C. M. Isada, and G. W. Procop. 2002. Rapid identification of Staphylococcus aureus and the mecA gene from BacT/ALERT blood culture bottles by using the LightCycler system. J. Clin. Microbiol. 40:2659-2661.

(12) Tan, T. Y., S. Corden, R. Barnes, and B. Cookson. 2001. Rapid identification of methicillin-resistant Staphylococcus aureus from positive blood cultures by real-time fluorescence PCR. J. Clin. Microbiol. 39:4529-4531.

1. A method for the detection of the presence of at least one of Staphylococcus aureus (SA), the antibiotic resistant forms thereof, and the antibiosis resistant forms of other bacteria comprising:
providing a sample;
extraction of nucleic acids from the sample;
bringing the extracted nucleic acids into contact with a first primer pair complementary to a targeted segment of the attBscc gene, a second primer pair complementary to a targeted segment of the mecA gene, and a third primer pair complementary to a targeted segment of the OrfX gene;
amplifying the targeted segment in each of the attBscc, mecA and OrfX genes for detection of a presence of the targeted segment if at least one of the three genes are present is the extracted nucleic acids from said sample; and
detecting the presence of the amplified targeted segments of nucleic acids to substantially simultaneously identify the presence of each of SA, the antibiotic resistant forms thereof, and the antibiotic resistant forms of other bacteria in the sample.

2. The method of claim 1, wherein the sample can be obtained from any source comprising biological cells.

3. The method of claim 1, wherein the nucleic acids comprise deoxyribonucleic acids (DNA).

4. The method of claim 1, wherein the antibiotic resistant form of SA comprise methicillin resistant Staphylococcus aureus (MRSA).

5. The method of claim 1, wherein the amplification of a targeted region in each of the attBscc, mecA and OrfX genes comprise running a real-time polymerase chain reaction procedure (real-time PCR).

6. The method of claim 5, wherein the real-time RT-PCR running conditions comprise 95° C. for 3 min, 35 cycles of 95° C. for 15 s and 60° C. for 30 s.

7. The method of claim 1 wherein prior to the step of amplifying the targeted segment in each of the attBscc, mecA and orfX genes, the method further comprises bringing the extracted nucleic acids into contact with a first labeled probe complementary to a targeted segment of the attBscc gene, a second labeled probe complementary to a targeted segment of the mecA gene, and a third labeled probe complementary to a targeted segment of the orfX gene.

8. The method of claim 7, wherein the first, second and third probes comprise the sequences SEQ ID NO. 4, SEQ ID NO. 7 and SEQ ID NO. 10 respectively.

9. The method of claim 8, wherein the detection of a targeted segment comprises detecting the presence of the respective probe's label.

10. The method of claim 9, wherein a fluorometer is used to detect the fluorescence emitted by the probe's label.

11. The method of claim 2, wherein the first primer pair comprises the sequences SEQ ID NO. 1 and SEQ ID NO. 2 or the sequences SEQ ID NO. 1 and SEQ ID NO. 3, the second primer pair comprises the sciences SEQ ID NO. 5 and SEQ ID NO. 6, and the third primer pair comprises the sequences SEQ ID NO. 8 and SEQ ID NO. 9.

12. A kit for the detection of the presence of at least one of SA, the antibiotic resistant forms thereof, said the antibiotic resistant forms of other bacteria in nucleic acids extracted from a sample, comprising:
means for exposing the extracted nucleic acids with a first primer pair complementary to a targeted segment of the attBscc gene, a second primer pair complementary to a targeted segment of the mecA gene, and a third primer pair complementary to a targeted segment, of the orfX gene.

13. The kit of claim 12, wherein the kit further comprises:
means for amplifying the targeted segment in each of the attBscc, mecA and orfX genes for detection of a presence of the targeted segment if at least one of the three genes are present in the extracted nucleic acids from said sample; and
means for detecting the presence of the amplified targeted segments of nucleic acids to substantially simultaneously identify the presence of each of SA, the antibiotic resistant forms thereof, and the antibiotic resistant forms of other bacteria in the sample.

14. The kit of claim 12, wherein the sample can be obtained from any source comprising biological cells.

15. The kit of claim 12, wherein the nucleic acids comprise deoxyribonucleic acids (DNA).

16. The kit of claim 12, wherein the antibiotic resistant form of SA comprise MRSA.

17. The kit of claim 12, wherein the first primer pair comprises the sequences SEQ ID NO. 1 and SEQ ID NO. 2 or the sequences SEQ ID NO. 1 and SEQ ID NO. 3, the second primer pair comprises the sequences SEQ ID NO. 5 and SEQ ID NO. 6, and the third primer pair comprises the sequences SEQ ID NO. 8 and SEQ ID NO. 9.

18. The kit of claim 12, wherein the kit further comprises means for bringing the extracted nucleic acids into contact with a first labeled probe complementary to a targeted segment of the attBscc gene, second labeled probe complementary to a targeted segment of the mecA gene, and a third labeled probe complementary to a targeted segment of the orfX gene, prior to amplifying the targeted segment in each of the attBscc, mecA and orfX genes.

19. The kit of claim 17, wherein the first, second and third probes comprise the sequences SEQ ID NO. 4, SEQ ID NO. 7 and SEQ ID NO. 10 respectively.
